(19) United States
(12) Patent Application Publication
(10) Pub. No.: US 2009/0227936 A1
(43) Pub. Date: Sep. 10, 2009

(54) Ultra bright LED induced tattoo removal

(76) Inventor: Frank Pellegrini, Dix Hills, NY (US)

Correspondence Address: Stephen E. Feldman, 12 East 41st Street, New York, NY, 10017, US

(21) Appl. No.: 12/381,134

(22) Filed: Mar. 6, 2009

Related U.S. Application Data
(60) Provisional application No. 61/068,369, filed on Mar. 7, 2008.

(51) Int. Cl. A61M 35/00; A61B 18/18

(52) U.S. Cl. 604/20; 606/9

(57) ABSTRACT

A device for removing tattoos is provided having ultra bright LEDs in a tight array to concentrate the output energy toward a skin area containing a tattoo. The output energy, such as, for a red color ultra bright LED, will be about 88 joules per square inch. The amount of energy emitted into the tattoo will penetrate the epidermis and into the dermis in which the tattoo is situated.

A method of removing tattoos using an optical device is provided that has a panel which houses a plurality of ultra bright LEDs. The ultra bright LEDs clusters are configured to penetrate the outer skin layer epidermis without damaging the skin by overheating and enter the next layer of skin, the dermis, to destroy the ink for effective tattoo removal.

### RELATED APPLICATIONS

This application claims the priority of provisional application Ser. No. 61/068,369, filed Mar. 7, 2008 and provisional application Ser. No. 61/941,173, filed Sep. 26, 2008.

### BACKGROUND OF THE INVENTION

1. Field of the Invention

This invention relates generally to removal of tattoos and other pigmented skin lesions without skin damage.

2. Description of the Related Art

A variety of medical procedures and techniques to remove tattoos are disclosed in the prior art. For example, dermabrasion has been used to remove tattoos, in which the skin is simply sliced off or abraded. This naturally produces scars. Often, pigments which lie in different skin layers are not removed along with the others. In addition to the scar, a dark shade from the remnants of the tattoo showing through remains. Another method involves the tangential excision and covering with a “split-skin graft”. The skin layer concerned is cut out under general anesthesia while it is tried to save as much as possible from the underlying skin layer. The open area is covered with split skin and saved from unnecessary scar formation over months by compression bandages, and adapted to the environment. More recent methods of removing tattoos include the use of LEDs using pulsed radiation and lasers. But these procedures and techniques also have many disadvantages. First, these procedures and techniques may produce “speckles” of relatively high power density that can cause significant local heating of tissues in general. Therefore, the heat generated by the procedures may damage skin tissues.

Second, the methods and procedure typically involve use of monochromatic light that have shorter duty cycle and may not be absorbed effectively by many dyes of varying colors. Third, the procedures for removing tattoos are not exactly affordable to every one. Fourth, the procedures cannot treat large surface areas and the treatment is focused on a very small area of a tattoo. Also, some of these tattoo treatments can be quite expensive.

For example, laser treatment entails delivering light energy to the tattoo in order to break the pigments into fragments which are then removed by the subjects' immune system. The drawbacks to laser removal are that the majority of the power of the laser is wasted as random heat must be removed to prevent tissue damage. Also, these treatments can be very expensive, painful, and not always effective. In addition, using lasers can cause reactions in certain of the chemicals used in the inks, therefore, leading to permanent darkening.

U.S. Patent Publication No. 2005/0148567, discloses a photosensitizer therapy method causing tattoo inks to fade or disappear. The photosensitizer is delivered intradermally (i.g. by injection) into the tattooed target tissue and irradicates the target tissue with energy to activate the photosensitizer. Although the invention uses a low energy LED light to activate the photosensitizer, the energy of the beam is insufficient to cause direct bond fracture in the dye molecules. This procedure simply uses the LED light to break down the photosensitizer molecule into new molecules which chemically interact with the dyes.

Similarly, U.S. Pat. No. 6,676,655 utilizes LED pulsing light in the near IR region to treat various dermatological conditions. Using pulsed radiation, however, can damage the skin due in part to the scattered light it causes and its ability to create surface heat.

All these disadvantages are addressed if ultra bright LED induced tattoo removal procedures are used without pulsed radiation. Ultra bright LED has no duty cycle, causes less skin damage, provides constant energy output and overcomes drawbacks associated with the prior art methods and procedures.

What is needed is a tattoo removal method and device, which is lower in cost, uses cool light sources and that can treat larger skin areas. What is particularly needed is a tattoo removal method and device that uses the singular properties of ultra bright LEDs without using pulsed radiation that penetrates through the outer skin without damaging said outer skin.

### SUMMARY OF THE INVENTION

A device for removing tattoos and pigmented lesions is provided having ultra bright LEDs in a tight array to concentrate the output energy toward a skin area containing a tattoo. The energy output generated during a five minute period by a high bright LED display will be about 88 joules per square inch. This amount of energy is capable of penetrating the epidermis and going into the dermis in which the tattoo is situated.

A method of removing tattoos using an optical device is provided that has a panel which houses a plurality of ultra bright LEDs. The ultra bright LEDs clusters are configured to provide maximum penetration of the outer skin layer (epidermis) without damaging the skin by overheating and penetrating into the next layer of skin, the dermis, where it will interact with the ink molecules leading to their destruction, thus resulting in effective tattoo removal.

As an example, the operator would place the apparatus approximately 1 to 2 inches above the tattooed area. The tattoo area is then exposed to the continuous light generated by the clustered ultra bright LEDs 26 for a specific period of time. In this particular example, the apparatus contains 120 ultra bright LEDs clustered in twelve rows of ten LEDs each. But the number may vary in other embodiments. The time the tattooed skin is exposed to the light of the ultra bright LEDs depends upon factors including the colors in the tattoo as well as its size. In this particular embodiment, however, the time is between 5 and 15 minutes. The average energy output, in a 15 minute session, would be 480 Joules. During this period of time the light would penetrate through the epidermis and into the dermal layer in which the tattoo resides. The absorption of the energy by the tattoo ink results in both heat generated in the ink molecules by molecular vibration and bond deformation by vibration, stretching and bending. The removal from the body of the by-products created by the destruction of tattoo inks can be facilitated by the increased blood supply and immune system response created by the application of L-Arginine or IRM compounds (immune response modifiers). The energy will break apart the bonds of the tattoo ink and cause it to be dispersed and absorbed into the body. By using the energy of the ultra bright LED, the tattoo will fade or be removed.

### BRIEF DESCRIPTION OF THE FIGURES

The above mentioned and other features, aspects and advantages of the present invention will become better understood with regard to following description, appended claims and accompanying drawings, wherein like reference numerals refer to similar parts throughout the several views where:

FIG. 1 is a perspective view of one embodiment of an optical device constructed in accordance with the present invention;

FIG. 2 is a perspective view of another device constructed in accordance with the present invention;

FIG. 3 is a perspective view of another optical device constructed in accordance with the present invention;

FIG. 4 is a block diagram showing various components which are used along with the device constructed in accordance with the present invention; and

FIG. 5 shows one embodiment of the present invention that uses a flexible neck in accordance with the present invention.

### DETAILED DESCRIPTION OF THE INVENTION

Although specific terms are used in the following description for sake of clarity, these terms are intended to refer only to particular structure of the invention selected for illustration in the drawings, and are not intended to define or limit the scope of the invention.

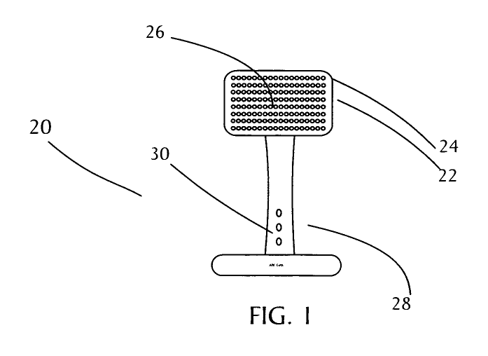

In one embodiment, referring to FIG. 1, an optical device 20, is provided, which includes a proximal end 22 that has an ultra bright LED panel 24. The ultra bright LED panel 24 houses a plurality of ultra bright LED cluster 26. In this particular embodiment, the device has a distal end 28 that has a control panel 30 that has switches that actuate, deactuate, and regulate the plurality of ultra bright LED cluster probes 26. The distal end is configured so that the LED panel 24 and the plurality of ultra bright LED cluster probes direct the panel.

The present invention involves using an ultra bright LED display that has an output energy of about 88 joules per square inch to remove tattoos. The ultra bright LEDs are configured to penetrate the outer skin layer epidermis without damaging the skin by overheating and enter the next layer of skin, the dermis, to destroy the ink for effective tattoo removal.

### EXAMPLES

It will be understood that the following embodiments of the present invention are intended to be illustrative of some of the possible applications or principles. Various modifications may be made by the skilled person without departing from the true spirit and scope of the invention.

### Example 1

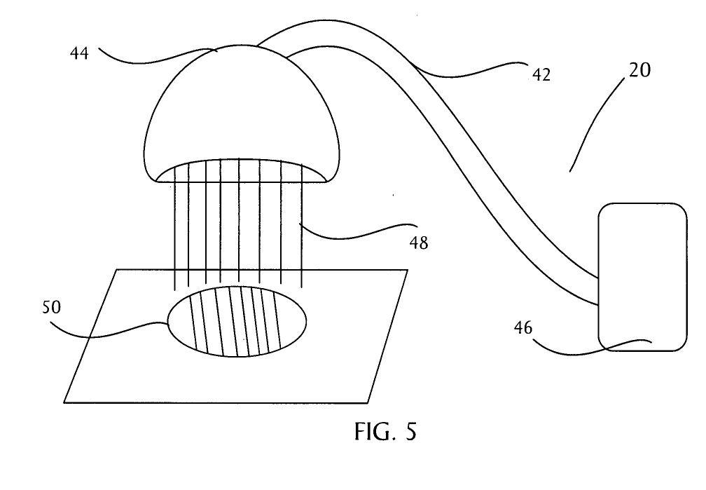

The operator places the apparatus approximately 1 to 2 inches above the tattooed area (See FIG. 5). The apparatus contains 120 ultra bright LEDs 26 clustered in twelve rows of ten LEDs each. The tattoo area is then exposed to the continuous light generated by the clustered ultra bright LEDs for 15 minutes. The average energy output, in this 15 minute session is 480 Joules. During this period of time, the light penetrates through the epidermis and into the dermal layer in which the tattoo resides. The absorption of the energy by the tattoo ink results in both heat generated in the ink molecules by molecular vibration and molecular bond deformation by vibration, stretching and bending. Thus, resulting in the tattoo being removed.

### Example 2

A thin layer of 10% to 15% of L-Arginine is applied directly to the tattoo area by the operator. The operator then places the apparatus approximately 1 to 2 inches above the tattooed area after L-arginine as been administered. The apparatus contains 120 ultra bright LEDs 26 clustered in twelve rows of ten LEDs each. The tattoo area is then exposed to the continuous light generated by the clustered ultra bright LEDs for 15 minutes. The average energy output, in this 15 minute session is 480 Joules. During this period of time, the light penetrates through the epidermis and into the dermal layer in which the tattoo resides. The absorption of the energy by the tattoo ink results in both heat generated in the ink molecules by molecular vibration and molecular bond deformation by vibration, stretching and bending. Thus, resulting in the tattoo being removed.

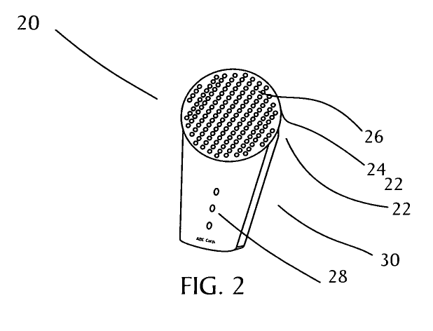

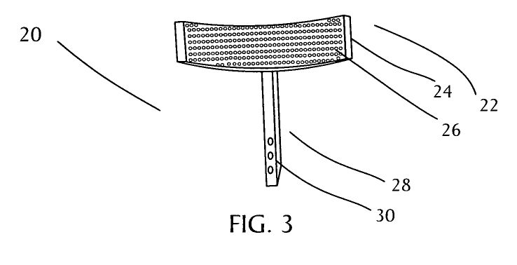

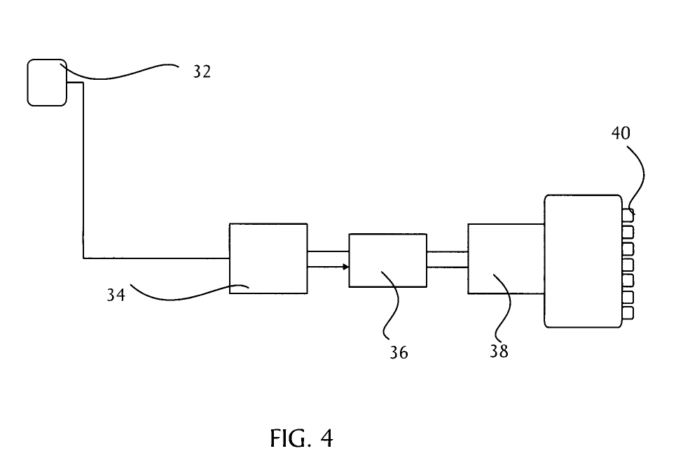

Referring to FIG. 2, a hand-held optical device 20 constructed in accordance with the present invention is illustrated. It is, however, understood, that the optical device 20 may be any different type or shape. The LED panel 24, in this embodiment, is circular in shape. Referring to FIG. 3, an optical device 20 constructed in accordance with the present invention is illustrated. The LED panel, 24 in this embodiment, is slightly concaved. In this one particular embodiment, the device 20 designed is for treating facial tattoos. The LED panel 24 may be advantageously shaped for treating facial tattoos of a person who is sitting in a chair. Referring to FIG. 4, a block diagram that shows various components that are used with an optical device constructed in accordance with the present invention are shown. The components are an AC power supply 32 that supplies power to an AC to DC converter 34 that is connected to a timer 36, a PCB (Printed Circuit Board) circuit 38 and ultra bright LED clusters 40 in series. The AC power supply 32 is converted to DC power supply by the AC to DC converter 34. The timer 36 that is connected in series to the converter 34 controls the time for which the ultra bright LED clusters 40 is in operation. The PCB circuit 38 is able to provide a variety of time and intensity settings for the timer 38 and ultra bright LED clusters 40. The time for which the ultra bright LED clusters are kept on may vary from case to case. Similarly, the intensity of the light produced by super bright LED clusters may vary and the number of ultra bright LED clusters that are in operation can be changed depending upon the requirement. The number of ultra bright LED clusters that are on is adjusted using the settings provided by the PCB circuit 38. The LED ultra bright clusters are configured to penetrate the outer skin layer without damaging said outer skin for effective tattoo removal. The average energy output, in a 15 minute session, would be 480 Joules. However, it is understood to one skilled in the art that the average energy output can vary depending on the length of the session.

Referring to FIG. 5, shows another embodiment of the present invention. FIG. 5 depicts a diagram that illustrates a use of a flexible neck in accordance with the optical device 20 in accordance with the present invention. A flexible neck 42 connects the lamp 44 containing the ultra bright LED clusters to a power board 46. The flexible neck advantageously allows the device 20 to be maneuvered and focuses the light 48 radiated by the ultra bright LED clusters on a tattooed area 50 with greater accuracy and flexibility.

### In Operation

The device works, without using pulsed radiation, by the energy contained in the light beam being absorbed by the tattoo ink dyes. This absorbed energy will result in an increased stretching, vibration and bending of the bonds which hold the dye (ink) molecules together. Ultimately, these bond stresses cause bond deformation with resulting bond failure. The frequencies chosen are those which produce energies which are absorbed by the bonds in the dyes but have minimal absorption by melanin in the skin or hemoglobin in the blood. Melanin and hemoglobin have maximum absorptions below 600 nm. Maximum absorption for melanin is 335 nm and for hemoglobin 310 nm.

For the light produced to be beneficial for removal tattoos, ultra bright LED's with high enough energy output are used. The output energy, such as, for a red color ultra bright LED (660-700 nm), will be about 88 joules per square inch. The brightness of light depends upon its photon density. The brighter the light, the greater will be its photon density. Since each photon at a given wavelength has the same energy, the greater the photon density, the greater will be the energy content of the light. Thus, using ultra bright LED's leads to a higher level of energy in the light beam. The wavelength of the ultra bright LEDs that are used is 660-700+nanometers. At 660 to 770 nm wavelength there is no light absorption by either melanin (skin coloring agent) or hemoglobin in blood. The greater the light intensity, the greater will be the energy content of the light. Heat is generated when the light is absorbed by the molecules thus increasing the molecular motion.

Melanin and hemoglobin do not absorb well at 660 to 700 nm of wavelength. Light would not generate heat on the skin at 660 to 700 nm of wavelength since the main chromophore of the skin is melanin but will generate heat on tattoo dyes since these dyes absorb energy in the 600-700 nm range of wavelength. Therefore, little to no heat is generated on the surface of the skin, but the light penetrates through the epidermis into the dermis where the tattoo ink is and the frequencies are absorbed by dye molecules causing increasing molecular motion and bond deformation. Ultra bright LEDs are from approximately 50 to 500 times brighter than standard LEDs. Thus, their energy content is likewise 50 to 500 times greater. The frequency of light used to destabilize the bonds in tattoo inks depends upon the composition of the ink and its color. The wavelength range may be between 400 to 900 nm.

In summary, the device is able to work for removing tattoos and pigmented lesions, by the energy contained in the light beam being absorbed by the tattoo ink dyes. This absorbed energy will result in an increased stretching, vibration and bending of the bonds which hold the dye (ink) molecules together. Ultimately, these bond stresses cause bond deformation with resulting bond failure. The output energy, such as, for a red color ultra bright LED, will be about 88 joules per square inch. The proximity of the bulbs and the amount of energy emitted into the tattoo will penetrate the epidermis and into the dermis in which the tattoo is situated.

In another embodiment, L-Arginine can be applied to the tattooed region before administering the method. It creates enlarged blood vessels which bring greater blood flow to the tattoo area. In addition, it creates an increase in the immune system response. These two mechanisms help speed up the removal of the by-products of the degradation of the tattoo dyes, thus, allowing for the tattoo to fade more quickly.

In one embodiment, an IRM (immune response modifier) compound can be applied. Specifically, IRM compounds containing L-Arginine can also increase the concentration of macrophages in the blood. Macrophages are specifically located in the lymph nodes and are white blood cells that phagocytizes necrotic cell debris and foreign material, including viruses, bacteria, and tattoo ink.

The IRM compound may be selected from a group consisting of imidazoquinoline amine; a tetrahydroimidazoquinoline amine; an imidazopyridine amine; a 1,2-bridged imidazoquinoline amine; a 6,7-fused cycloalkylimidazopyridine amine; animidazonaphthyridine amine; a tetrahydronaphthyridine amine; an oxazoloquinoline amine; a thiazoloquinoline amine; an oxazolopyridine amine; a thiazolopyridine amine; an oxazolonaphthyridine amine; a thiazolonaphthyridine amine; or a 1H-imidazodimer fused to a pyridine amine, a quinoline amine, a tetrahydroquinoline amine, a naphthyridine amine, and a tetrahydronaphthyridine amine.

The foregoing description of the preferred embodiments of the present invention has been presented for the purpose of illustration and description. It is not intended to be exhaustive or to limit the invention to the precise form disclosed. Many modifications and verifications are possible in light of the above teaching. It is intended that the scope of the invention be limited not by this detailed description, but rather by the claims appended hereto. Since many embodiments of the present disclosure can be made without departing from the spirit and scope of the present invention, the present invention resides in the claims hereafter appended.

1. An optical device for removing tattoos and pigmented lesions that comprises:
a panel at a proximal end;
said panel comprising a plurality of ultra bright LEDs; and
wherein said ultra bright LEDs are configured so that induced LED energy irradiates a tattooed skin without collateral skin damage.

2. The optical device of claim of I further comprising:
a control panel at a distal end; and
said control panels having a plurality of controls for regulating said plurality of ultra light LEDs.

3. The optical device of claim 1 wherein said plurality of ultra bright LEDs are of visible light.

4. The optical device of claim 1 wherein said plurality of ultra bright LEDs are red LEDs.

5. The optical device of claim 1 wherein said plurality of ultra bright LEDs are of a combination of visible and infra red LEDs.

6. The optical device of claim 1 wherein said device is a hand-held device.

7. An optical device for removing tattoos and pigmented lesions that comprises:
a panel at a proximal end;
said panel comprising a plurality of ultra bright LEDs; and
L-arginine,
wherein said ultra bright LEDs are configured so that induced LED energy irradiates a tattooed skin without collateral skin damage.

8. A method of removing tattoos and pigmented lesions using an optical device comprising:
treating a tattooed region comprising administering a plurality of ultra bright LEDs to a subject, so that tattoo removal is accomplished by irradiating a tattooed region.

9. The method of claim 8, wherein L-Arginine is applied to the tattooed region before administering the method.
